(19) United States
(12) Patent Application Publication
(10) Pub. No.: US 2019/0249225 A1
(43) Pub. Date: Aug. 15, 2019

(54) DISPENSER MAPPING APPARATUS FOR MULTI-ANALYSIS AND THE OPERATION METHOD THEREOF

(71) Applicant: GENOPLAN KOREA INC., Seoul (KR)

(72) Inventors: Soung Sik KIM, Bucheon-si (KR); Ji Hyun CHUNG, Seoul (KR); Min Jin KIM, Yongin-si (KR); Jung Min KIM, Seoul (KR); Byung Kyu KANG, Seongnam-si (KR)

(21) Appl. No.: 16/199,028

(22) Filed: Nov. 23, 2018

(30) Foreign Application Priority Data
Feb. 9, 2018 (KR) 10-2018-0016555

(51) Int. Cl. C12Q 1/6806; C12Q 1/6876; G06F 7/02; C12Q 1/6837; C12Q 1/686; B01L 3/00

(52) U.S. Cl. CPC C12Q 1/6806; C12Q 1/6876; G06F 19/702; G06F 19/20; B01J 2219/00306; G06F 7/023; C12Q 1/6837; C12Q 1/686; B01L 3/5025; G06F 19/22

(57) ABSTRACT

Provided is a dispenser mapping apparatus for multi-analysis and an operation method thereof according to an embodiment. The method may include receiving analysis information—the analysis information including information relating to at least one of a plurality of samples, a detection gene for each of the plurality of samples, and a primer corresponding to the detection gene which are subject to analysis—dividing a reaction plate into a plurality of areas and sequentially arranging the plurality of samples and the plurality of primers on the plurality of areas of the reaction plate.

### CROSS-REFERENCE TO RELATED APPLICATIONS

This application claims the priority to Korean patent application No. 10-2018-0016555 filed on Feb. 9, 2018, the entire disclosure of which is hereby incorporated by reference.

### BACKGROUND

The present disclosure provides a dispenser mapping apparatus for multi-analysis and an operation method thereof, and in particular, a dispenser mapping apparatus capable of designing a sequence and combination of dispensing that is optimized for analysis information in multi-analysis and an operation method thereof.

### FIELD OF THE DISCLOSURE

Polymerase chain reaction (PCR) exponentially amplifies a nucleic acid having a certain base sequence segment by serially copying the segment having the certain base sequence of the nucleic acid by repeatedly heating and cooling a sample solution containing the nucleic acid. PCR is widely used in life science, genetic engineering and medical fields, etc. for the purposes of analysis and diagnosis.

However, an analysis based on such amplification of nucleic acid or a detection method to detect a certain gene is limited in that one template may be searched one at a time. For example, suppose a patient develops the same symptom as before. In this case, even though the patient may show the same symptom as before, there may be many cases where the patient's illness may have been caused by various types of infectious agents. Therefore, diagnosis of various pathogenic organisms will be needed one by one. However, amplifying each template one by one in a situation where several templates need to be amplified is cumbersome and very time-consuming. Also, the amount of nucleic acid that can be extracted from a limited sample under general environment is finite or limited. Therefore, it may often be the case that repeated diagnosis is made impossible through amplification of nucleic acid using a limited amount of nucleic acid.

To solve the aforementioned problem, a multi-analysis method for simultaneously analyzing nucleic acids of many templates from one or more samples was introduced.

Generally, such multi analysis is done by sequentially dispensing a plurality of samples, primers, reagents, etc., to a reaction plate using a dispensing apparatus having a plurality of nozzles. The more the samples or the nucleic acids to detect, the more time it takes to carry out dispensing. Therefore, in multi-analysis, how to effectively design dispensing sequence or combination of such sample, primer, etc. is one of the most important factors in determining the overall time for analysis.

As such, depending on the number of samples or nucleic acids to detect, etc., there is an increase in demand for a technology that designs the sequence and combination of optimal dispensing. However, such technology is almost non-existent to date.

### DETAILED DESCRIPTION OF DISCLOSURE

### Problem

The present disclosure provides, in order to overcome the problem, a dispenser mapping apparatus for multi-analysis capable of designing dispensing sequence and combination of sample, primer, etc., optimized for analysis information in multi analysis, and an operation method thereof.

The problem is not limited to the above, and other problems that have not been mentioned herein will be clearly understood by a person of ordinary skill in the art by the following disclosures.

### Technical Solution

In an embodiment, a method for operating a dispenser mapping apparatus for multi-analysis is provided. The method may include receiving analysis information, the analysis information including information relating to at least one of a plurality of samples, a detection gene for each of the plurality of samples, and one of a plurality of primers corresponding to the detection gene which is subject to analysis, dividing a reaction plate into a plurality of areas and sequentially arranging the samples and the primers on the divided areas of the reaction plate.

The dividing into the plurality of areas may be performed based on at least one of a maximum possible simultaneous dispensing count, an interval between dispensing nozzles and an interval between wells of the reaction plate.

At least one of rows and columns of each of the areas divided from the reaction plate may be identical to the maximum possible simultaneous dispensing count.

The method may further include grouping at least one of the plurality of samples and the primers based on the analysis information into at least one group, wherein the arranging of the plurality of samples and the primers is performed based on a result of the grouping, wherein a number of maximum samples and primers belonging to each of the groups is identical to a maximum possible simultaneous dispensing count.

The analysis information may further include information relating to arrangement of the primers on a primer plate, wherein the grouping groups the primers into at least one group by sequentially giving priority to a dispensing count of each of the primers and an arrangement sequence of the primer plate.

The method may further include calculating a simultaneous dispensing count with respect to at least one of the samples and the primers belonging to each of the groups, wherein the arranging of the sample and the primers is performed by sequentially arranging at least one of the samples and the primers belonging to each of the groups in the divided areas of the reaction plate corresponding to the simultaneous dispensing count.

The arranging of the samples and the primers may preferentially arrange samples and primers belonging to a group with a great number of the simultaneous dispensing count.

The method may further include calculating a simultaneous dispensing count and a single dispensing count with respect to at least one of the samples and the primers belonging to each of the groups, wherein the arranging the samples and the primers includes sequentially arranging at least one of the samples and the primers belonging to each of the groups in the divided areas of the reaction plate corresponding to the simultaneous dispensing count and sequentially arranging, when an arrangement according to the simultaneous dispensing count is completed, at least one of the samples and the primers belonging to each of the groups in remaining areas of the reaction plate based on the single dispensing count.

The analysis information may further include information relating to an arrangement of the samples on a sample plate and an arrangement of the primers on a primer plate, wherein the method may further include arranging the primers on a mixing plate based on the result of the grouping and mutually mapping a sample arrangement of the sample plate and the reaction plate and a primer arrangement of the primer plate, the mixing plate and the reaction plate.

When the mapping is completed, at least one of the grouping result, the simultaneous dispensing count, the single dispensing count, the samples with respect to the reaction plate, the arrangement of the primers and the mapping result may be stored into a storage part corresponding to the analysis information.

The method may further include recommending the samples suitable for the analysis information and the arrangement of the primers on the reaction plate according to a predetermined standard in response to receipt of the analysis information, wherein the standard includes at least one of dispensing time, a number of replacement of dispensing nozzles and moving distance of the dispensing nozzles.

In an embodiment, a computer readable recording medium recording a program to perform the method is provided.

In an embodiment, a dispenser mapping apparatus for multi analysis is provided. The apparatus may include an input part receiving analysis information—the analysis information including information relating to at least one of a plurality of samples, a detection gene for each of the plurality of samples, and one of a plurality of primers corresponding to the detection gene which is subject to analysis—and an arrangement part dividing a reaction plate into a plurality of areas and sequentially arranging the samples and the primers on the divided areas of the reaction plate.

The arrangement part may divide the reaction plate into a plurality of areas based on at least one of a maximum possible simultaneous dispensing count, an interval between dispensing nozzles and an interval between wells of the reaction plate.

At least one of rows and columns of each of the divided areas of the reaction plate may be identical to the maximum possible simultaneous dispensing count.

The apparatus may further include a cluster part grouping at least one of the plurality of samples and the primers into at least one group based on the analysis information, wherein the arrangement part performs arrangement of the plurality of samples and the primers based on a result of the grouping, wherein a number of maximum samples and primers belonging to each of the groups is identical to a maximum possible simultaneous dispensing count.

The analysis information may further include information relating to an arrangement of the primers on a primer plate, wherein the cluster part groups the primers into at least one group by sequentially giving priority to a dispensing count of each of the primers and an arrangement sequence of the primer plate

The apparatus may further include a calculation part calculating a simultaneous dispensing count with respect to at least one of the samples and the primers belonging to each of the groups, wherein the arrangement part sequentially arranges at least one of the samples and the primers belonging to each of the groups in the divided areas of the reaction plate corresponding to the simultaneous dispensing count.

The arrangement part may preferentially arrange sample and primer that belong to a group with a great number of the simultaneous dispensing count.

The apparatus may further include a calculation part calculating a simultaneous dispensing count and a single dispensing count with respect to at least one of the samples and the primers belonging to each of the groups, wherein the arrangement part sequentially arranges at least one of the samples and the primers belonging to each of the groups in the divided areas of the reaction plate corresponding to the simultaneous dispensing count, and sequentially arranges, when an arrangement according to the simultaneous dispensing count is completed, at least one of the samples and the primers belonging to each of the groups in remaining areas of the reaction plate based on the single dispensing count.

The analysis information may further include information relating to an arrangement of the samples on a sample plate and an arrangement of the primers on a primer plate, wherein the apparatus further includes a mapping part arranging the primers on a mixing plate based on the result of the grouping and mutually mapping a sample arrangement of the sample plate and the reaction plate and a primer arrangement of the primer plate, the mixing plate and the reaction plate.

The apparatus may further include a storage part storing, when the mapping is completed, at least one of the grouping result, the simultaneous dispensing count, the single dispensing count, the samples with respect to the reaction plate, the arrangement of the primers and the mapping result corresponding to the analysis information.

The apparatus may further include a recommendation part recommending the samples suitable for the analysis information and the arrangement of the primers on the reaction plate according to a predetermined standard in response to receipt of the analysis information, wherein the standard includes at least one of dispensing time, a number of replacement of dispensing nozzles and moving distance of the dispensing nozzles.

### Effects of the Disclosure

The present disclosure provides that designing of dispensing sequence and combination each optimized for multi analysis involving different number of samples and detection genes can be easily carried out.

Also, the present disclosure provides that by dividing a reaction plate into a plurality of areas and arranging sample and primer for dispensing in consideration of the number of possible simultaneous dispensing of a dispensing unit, the space of the reaction plate may be more effectively utilized.

Also, the present disclosure provides that by categorizing sample or primer into one or more groups capable of simultaneously dispensing in one unit and arranging it on a reaction plate in a group unit based on the number of simultaneous dispensing, etc., the number of replacement of dispensing nozzles, moving distance, dispensing time, etc. in performing analysis may be optimized.

Also, the present disclosure provides that by optimizing the sequence and combination of dispensing for multi analysis, the running cost of analysis apparatus can be reduced.

Also, the present disclosure provides that by making arrangement of sample and primer regarding reaction plate and storing mapping result between plates and putting them in a database, dispensing design function suitable for prompt analysis information may be provided to users.

### BRIEF DESCRIPTION OF THE DRAWINGS

Brief description on each drawing figure will be provided so that the drawing figures referenced in the detailed description may be more sufficiently understood

FIG. 1 illustrates a reaction plate in an embodiment.

FIG. 2 illustrates a sample plate and a primer plate in an embodiment.

FIG. 3 illustrates a dispenser mapping apparatus in an embodiment.

FIG. 4 illustrates an operation method of a dispenser mapping apparatus in an embodiment.

FIG. 5 illustrates an operation method of a dispenser mapping apparatus in an embodiment.

FIGS. 6 to 13 illustrate exemplary operations of a dispenser mapping apparatus in an embodiment.

### DETAILED DESCRIPTION OF THE PREFERRED EMBODIMENTS

Hereinafter, example embodiments will be described with reference to the accompanying drawings; however, for reference numerals, with respect to the same elements, even though they may be displayed in different drawings, such elements use same reference numerals as much as possible. Also, in explaining the example embodiments, detailed description on known elements or functions will be omitted if it is determined that such description will interfere with understanding of the embodiments. In addition, the example embodiments may be embodied in different forms and should not be construed as limited to the embodiments set forth herein but may be modified and variously implemented by those skilled in the art.

In the drawing figures, dimensions may be exaggerated for clarity of illustration. It will be understood that when an element is referred to as being “between” two elements, it can be the only element between the two elements, or one or more intervening elements may also be present. It will be understood that when a part includes or has an element, it does not mean that other elements are excluded but that other elements may be further included. Also, in explaining elements, terms like “first”, “second”, “A”, “B”, “(a)”, “(b)”, etc. may be used. However, such terms are used to distinguish one from the others only and they do not affect the essence, nature, sequence, order, etc.

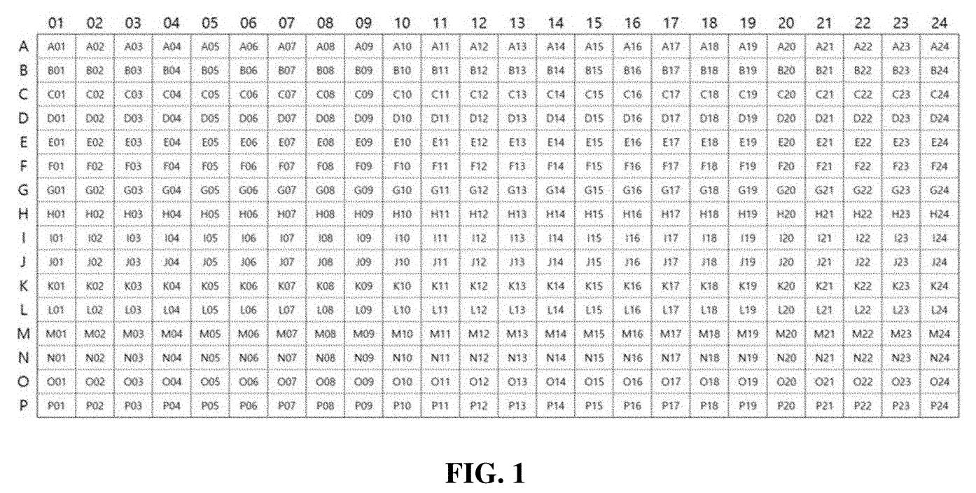

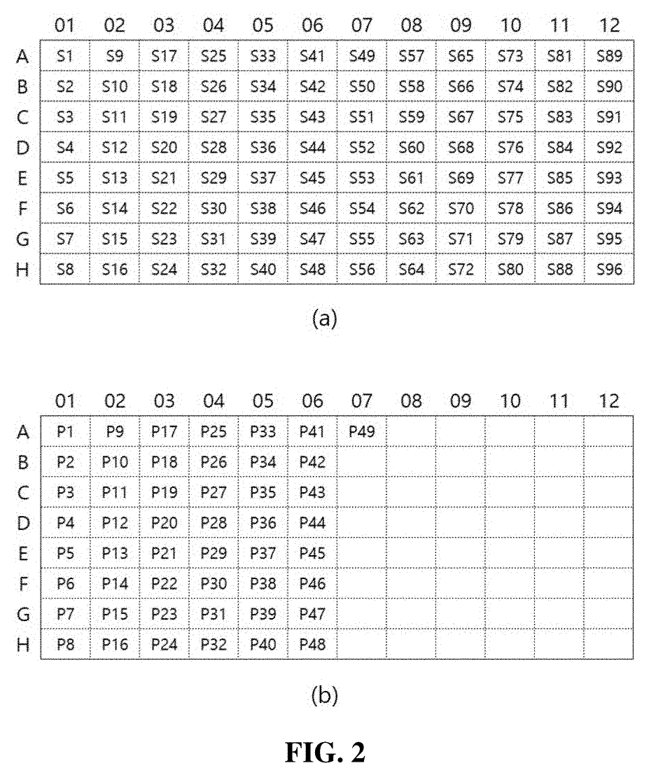

FIG. 1 illustrates a reaction plate in an embodiment. FIG. 2A illustrates a sample plate in an embodiment. FIG. 2B illustrates a primer plate in an embodiment.

In the present disclosure, the reaction plate is a plate where a predetermined reaction (e.g., polymerase chain reaction (PCR), etc.) occurs by mixing a primer, a reagent, etc. to each of a plurality of samples for multi analysis. As shown in FIG. 1, the reaction plate may include 384 wells (16 rows×24 columns) where the reaction may be performed. The plurality of wells that make up the reaction plate, as shown in FIG. 1, may be coordinates by the combination of rows and columns, for example, as A01, B01, C01, . . . , etc.

Each of a sample plate and a primer plate may refer to a plate in which a plurality of samples that include one or more genes, which are subject for a detection, and a plurality of primers that correspond to a gene which is subject for the detection are arranged. Here, the samples may include, for example, blood, saliva, secretion, cells, etc. collected from humans, animals, etc., and primers serves as a starting point for DNA synthesis and may be made of base sequence complementary to certain sequence of detection target gene.

The sample plate and the primer plate may include 96 wells (8 rows×12 columns). For example, as shown in FIG. 2, the sample plate may be sequentially arranged where 96 samples are numbered S1, S2, . . ., etc. and arranged in a direction from a left column to a right column, and similarly, the primer plate may be sequentially arranged where 49 primers that correspond to the genes, which are subject for a detection, are numbered P1, P2, . . . , etc. and arranged in the direction from the left column to the right column. Furthermore, the wells of the sample plate and the primer plate may be coordinates as, for example, A01, B01, C01, . . . , etc. based on the combination or the rows and columns as in the case of the reaction plate.

However, such reaction plate, sample plate and primer plate are exemplary only, and depending on an embodiment to which the present disclosure is applied, the number of wells, arrangement, etc. of each plate may change based on the settings, etc. by a user.

Though not shown, transfer of a sample and/or a primer between a sample plate and a reaction plate, and/or between a primer plate and a reaction plate may be performed via a dispensing unit. Such dispensing unit may include a plurality of dispensing nozzles that are consecutively arranged in one direction, and it may be configured such that such dispensing nozzles are arranged in wells of each plate to transfer a sample, a primer, etc.

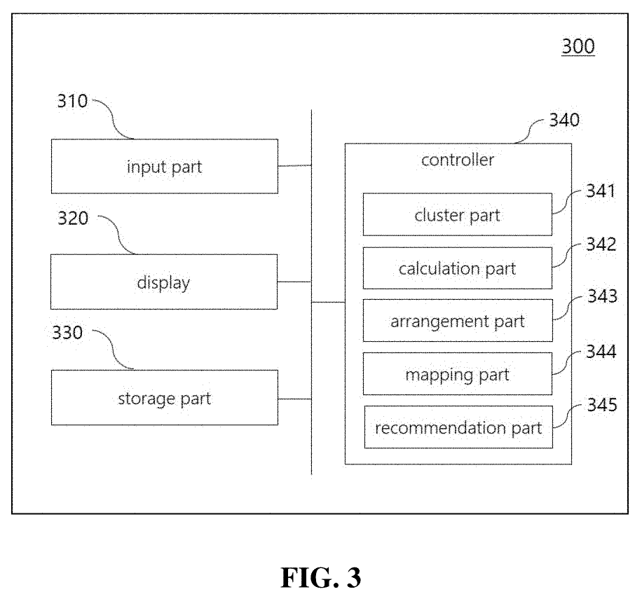

The present disclosure provides design of dispensing sequence and combination that are optimized for multi analysis of variant sample and gene via sample plate, primer plate, reaction plate, virtual dispensing unit, etc., corresponding to the plate, dispensing/analysis apparatus that are actually used by users. The details will be described with reference to FIGS. 3 to 13 below.

FIG. 3 illustrates a dispenser mapping apparatus in an embodiment.

Referring to FIG. 3, a dispenser mapping apparatus 300 may include an input part 310, a display 320, a storage part 330 and a control part 340.

The input part 310 may receive various data from a user of the dispenser mapping apparatus 300. In particular, in the dispenser mapping apparatus 300 in the present disclosure, the input part 310 may receive analysis information for multi analysis from the user. Here, the analysis information may include information relating to at least one of a plurality of samples, a detection gene for each of the plurality of samples, and one of a plurality of primers corresponding to the detection gene, arrangement of the plurality of samples on the sample plate and arrangement of the plurality of primers on the primer plate which are subject to analysis. Furthermore, the data may further include information relating to the number of wells of each plate, arrangement, etc. and information relating to reagents used for reaction, dispensing units, etc. but they are limited thereto. The input part 310 may employ various input devices that can be applied in the applicable field of technology such as a key, a touch panel, a touch screen, etc.

The display 320 may display an image or data supplied from a control part 340 (for example, arrangement information of a sample and/or primer with respect to each plate, mapping information between plates, etc.) onto a screen and provide user interface for user's input that is required for operation of the dispenser mapping apparatus 300. The display may be a liquid crystal display (LCD), an organic light emitting diode (OLED), an active matrix organic light emitting diode (AMOLED), etc.

In the storage part 330, various data may be stored which entails operation of the dispenser mapping device 300. The data may include analysis information for performing multi analysis, arrangement information of a sample and/or a primer with respect to each plate, arrangement mapping information between plates, etc., but is not limited thereto. The storage part 330 may be implemented with a storage device in various forms where information input/output is possible such as, as known to a person of ordinary skill in the art, a hard disk drive (HDD), a read only memory (ROM), a random access memory (RAM), an electrically erasable and programmable read only memory (EEPROM), a flash memory, a compact flash (CF) card, a secure digital (SD) card, a smart media (SM) card, a multimedia card (MMC), a memory stick, etc. The storage part 330 may be provided inside the dispenser mapping apparatus 300 or in a separate device.

The controller 340 may control an overall operation of the dispenser mapping apparatus 300 and signal flow between inner elements thereof. The controller 340 may also perform a data processing function to process data. As such, the controller 340 may include a cluster part 341, a calculation part 342, an arrangement part 343, a mapping part 344 and a recommendation part 345.

The cluster part 341 may group at least one of a plurality of samples and primers into at least one group based on analysis information. Here, the maximum number of samples and/or primers belonging to each group may be the same as the maximum possible number of simultaneous dispensing of a dispensing unit. For example, such grouping may be performed by sequentially arranging the samples or primers on a virtual plate based on the number of dispensing the samples or primers, arrangement sequence on each plate, etc. The group divided by the cluster part 341 may be simultaneously arranged (or dispensed) on a reaction plate or a mixing plate at least one time as one dispensing unit.

The calculation part 342 may calculate the number of simultaneous dispensing and/or single dispensing for each group with respect to grouped samples and/or primers by the cluster part 341. Here, the number of simultaneous dispensing may refer to the possible number of dispensing all of the samples or primers belonging to the same group simultaneously on a reaction plate, etc. via the dispensing unit, and the number of single dispensing may refer to the number of dispensing sample or primer singly dispensed after simultaneous dispensing.

The arrangement part 343 may divide a reaction plate into a plurality of areas. For example, the arrangement part 343 may divide the reaction plate into a plurality of areas based on at least one of the possible number of maximum simultaneous dispensing of a dispensing unit, the space between dispensing nozzles and the space between a plurality of wells that form the reaction plate. Here, at least one of rows and columns of the divided areas may be the same as the number of possible maximum simultaneous dispensing of the dispensing unit.

In addition, the arrangement part 343 may position the primers to a mixing plate based on a result of grouping the primers by the cluster part 341 or sequentially arrange a plurality of samples and primers to the divided areas of the reaction plate based on the number of simultaneous dispensing and/or of single dispensing of the samples and/or the primers belonging to each group calculated by the calculation part 342.

The mapping part 344 may, once arrangement of the samples and primers with respect to the reaction plate is completed, mutually map a sample arrangement between the sample plate and the reaction plate; and a primer arrangement among the primer plate, the mixing plate, and the reaction plate. Such mapping may be performed by mutually connecting the coordinates of the wells where the same samples and primers are arranged in each plate. Such mapping result may be provided to a user, and the user will perform multi analysis on the samples by controlling actual dispensing or the dispensing apparatus based on such mapping result.

The recommendation part 345 may recommend an arrangement of the plurality of samples and the plurality of primers suitable for analysis information on the reaction plate in response to receipt of analysis information. In particular, the recommendation part 345 may search arrangement of the plurality of samples and the plurality of primers suitable for analysis information by searching the storage part 330 and may provide it to a user. Here, the user may be provided with a dispensing sequence and a combination suitable for his or her own analysis based only on the simple method of selecting one among the recommended arrangements.

Though not shown in FIG. 3, the dispenser mapping apparatus 300 may further include a communication part. The communication part is provided for direct connection with outside or for connection through a network and may be a wire and/or wireless communication part. In particular, the communication part may transfer, by wire or wireless, data (for example, information on arrangement of the samples and primers with respect to the reaction plate, etc.) from the controller 340, the storage part 330, etc., or may receive data from outside via wire or wireless and transfer it to the controller 340 or store it in the storage part 330. In particular, the communication part may receive analysis information and transfer it to the controller 340 and the storage part 330. Here, the communication part may perform a function as the input part 310 that receives analysis information. The communication part may communicate via LAN, Wideband Code Division Multiple Access (WCDMA), Long Term Evolution (LTE), Wireless Broadband Internet (WiBro), Radio Frequency (RF), Wireless Lan, Wireless Fidelity, Near Field Communication (NFC), Bluetooth, Infrared (IR), etc. However, the list is exemplary only, and other wire/wireless communication technology may be used.

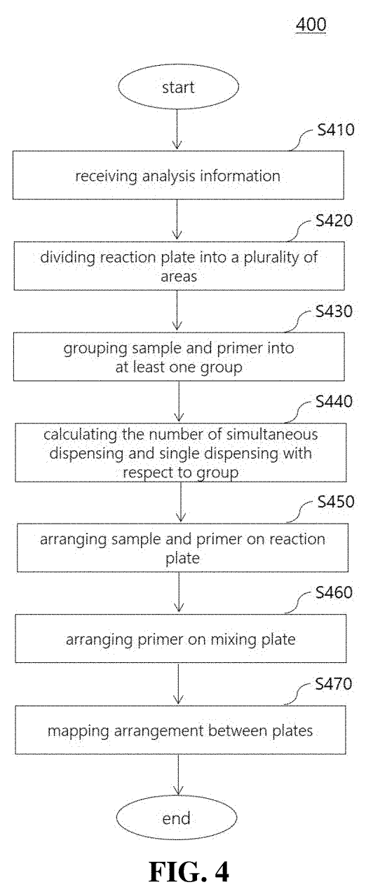

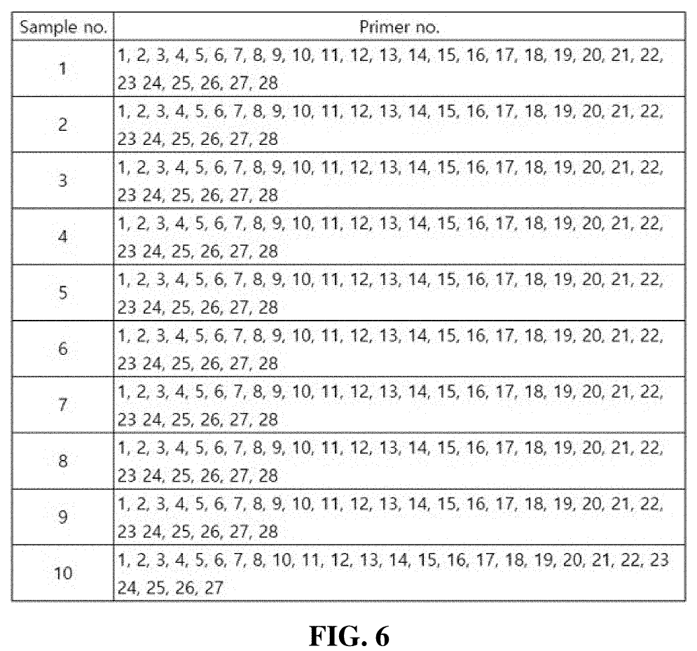

FIG. 4 illustrates an operation method of a dispenser mapping apparatus in an embodiment. FIGS. 6 to 13 illustrate exemplary operation of a dispenser mapping apparatus in an embodiment.

In S410, the input part 310 may receive analysis information from a user. Here, the analysis information may include information relating to at least one of a plurality of samples, a detection gene for each of the plurality of samples, one of a plurality of primers corresponding to the detection gene, arrangement of the samples on a sample plate and arrangement of the primers on a primer plate, which are subject to analysis. Such analysis information may be directly input from a user via the input part 310 or received from a user terminal connected by wire or wireless communication.

For example, but without any limitation thereto, the analysis information, as shown in FIG. 6, may be input or provided as a list of primers corresponding to one or more genes to detect with respect to each of the plurality of samples. Here, the detection genes and primers corresponding to each sample may be identical to or different from each other at least in part.

Also, in an embodiment, for detecting a gene that is identical to the entire sample, the analysis information that is received in S310 may include information relating to the total number of samples and detection genes. That is, in this case, a user may receive dispensing design suitable for multi analysis of the applicable sample just by inputting the number of the entire samples and the detection genes.

In S420, the arrangement part 343 may divide the reaction plate into a plurality of areas. S420 may be performed based on at least one of the number of possible maximum simultaneous dispensing, the space between dispensing nozzles and the space between a plurality of wells that form the reaction plate. Here, at least one of rows and columns of the divided areas may be the same as the number of maximum possible simultaneous dispensing of the dispensing unit.

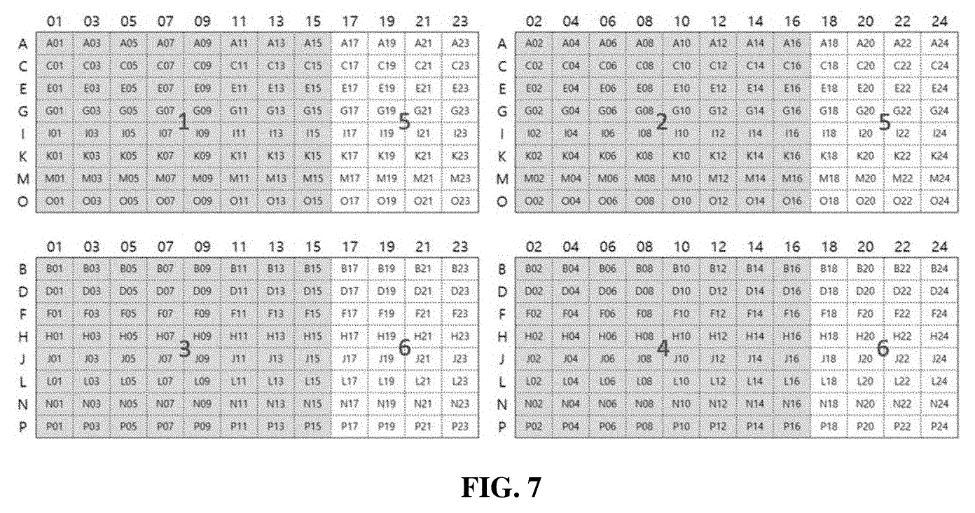

For example, referring to FIG. 7, the number of the maximum possible simultaneous dispensing of the dispensing unit is 8, and the reaction plate includes 384 wells (16 rows×24 columns). If the distance between the dispensing nozzles is twice the distance between the wells of the reaction plate, the arrangement part 343 may divide the reaction plate into four areas (8 rows×12 columns) by alternating the rows and columns of the wells for the first time. Here, the areas that are divided for the first time may have 8 rows which is identical to the number of maximum simultaneous possible dispensing. Thereafter, the arrangement part 343 may divide the columns in the areas that were divided for the first time by 8 areas based on the number of maximum simultaneous possible dispensing, and, as shown in FIG. 7, six areas (first to sixth areas) may be divided as sections.

Meanwhile, the information which becomes the basis for performing S420 such as the number of maximum possible simultaneous dispensing, etc., may be received from a user along with analysis information or stored as default in the storage part 330.

In S430, the cluster part 341 may group at least one of a plurality of samples and primers into at least one group based on analysis information. As described above, the number of maximum samples and/or primers belonging to each group may be the same as the number of maximum possible simultaneous dispensing of the dispensing unit.

In an embodiment, the cluster part 341 may group a plurality of primers into one or more groups by employing the method of arranging on a virtual plate by sequentially giving a priority to an arrangement sequence of the plurality of primers on the primer plate and the dispensing counts of each of the plurality of primers.

For example, referring to FIGS. 2B and FIG. 6, the primers included in the analysis information have different arrangement sequences on the primer plate, and at least a portion of the primers may be different from primers with different dispensing counts. That is, as shown in FIG. 6, since two primers P9 and P28 are not applied to sample 10, the two primers may have 9 dispensing counts unlike the primers with 10 dispensing counts.

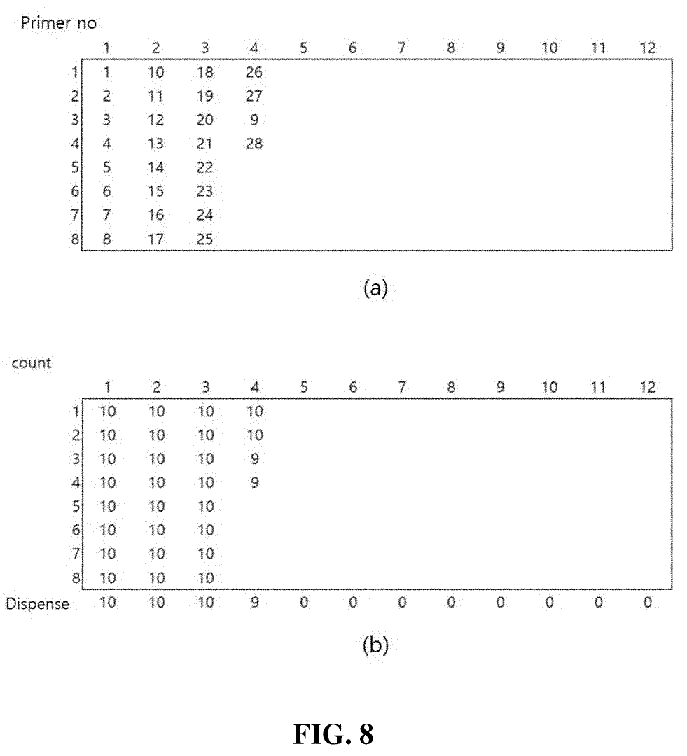

Here, the cluster part 341 may, as shown in FIG. 8A, preferentially arrange the primers with 10 dispensing counts beginning from a first column on a virtual plate that has the same number of rows as the maximum possible simultaneous dispensing counts (for example, 8) of the dispensing unit and thereafter, the primers with 9 dispensing counts may be positioned. As such, the primers may be grouped. Here, between the primers with the same dispensing counts, those primers that have fast/early arrangement sequence on the primer plate may be preferentially arranged on the virtual plate. Accordingly, the primers may be grouped into four groups as shown in FIG. 8A.

Meanwhile, the group that is divided in S430 is one dispensing unit, and as described below, simultaneous arrangement (or dispensing) may be performed at least one time on the reaction plate or the mixing plate.

In S440, the calculation part 342 may calculate the simultaneous dispensing counts and single dispensing counts with respect to at least one of the samples and primers belonging to each group.

For example, referring to FIG. 8A and FIG. 8B, the primers belonging to first to third groups from the left, may have only 10 simultaneous dispensing counts. On the other hand, the primers belonging to the third group may have 9 simultaneous dispensing counts and one single dispensing count for each of the 2 primers P26 and P27.

In S450, the arrangement part 343 may sequentially arrange a plurality of samples and primers on the reaction plate divided into a plurality of areas based on the number of simultaneous dispensing and/or the number of single dispensing calculated in S440.

In particular, the arrangement part 343 may sequentially arrange at least one of the samples and primers belonging to each group corresponding to the simultaneous dispensing counts on the divided areas of the reaction plate. Here, the arrangement may be preferentially performed with respect to the samples and/or primers belonging to a group with more simultaneous dispensing counts. Also, S450 may be performed as follows: once arrangement according to the simultaneous dispensing counts is complete, based on the single dispensing counts, at least one of the samples and the primers belonging to each group may be sequentially arranged in the remaining areas of the reaction plate.

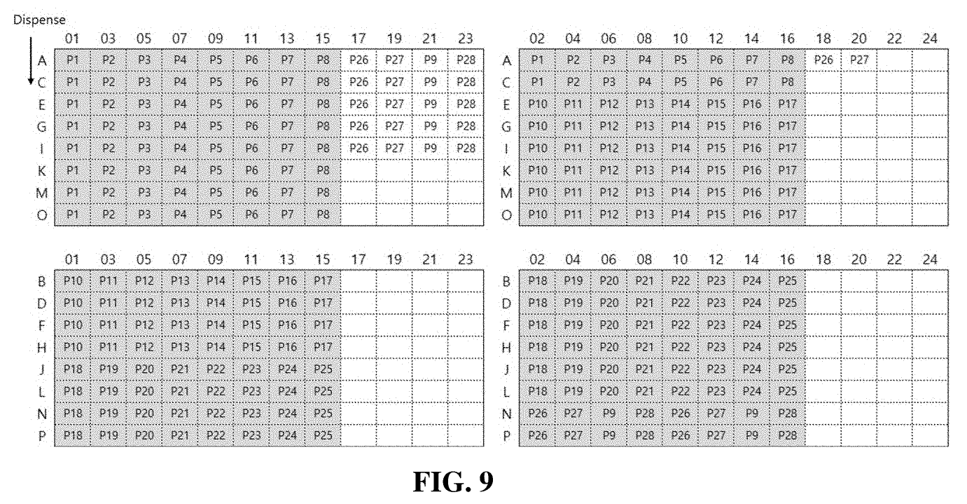

For example, referring to FIGS. 8 and 9, the arrangement part 343 may arrange primers P1 to P8 belonging to a first group 10 times from a first row in a first area to a second row in a second area of a reaction plate corresponding to the simultaneous dispensing counts, thereafter, in the same way, arrange primers P10 to P17 belonging to a second group and primers P18 to P25 belonging to a third group 10 times from a third row in the second area to a 6th row in a fourth area of the reaction plate, thereafter arrange primers P26, P27, P9, and P28 belonging to the fourth group 9 times in the remaining areas of the fourth area and a fifth area of the reaction plate corresponding to the simultaneous dispensing counts. As such, arrangement based on the number of simultaneous dispensing may be performed. Also, once the arrangement based on the simultaneous dispensing is complete, the arrangement part 343 may thereafter arrange two primers P26 and P27 of the fourth group having single dispensing counts into the remaining areas of the fifth area, thereby completing the arrangement of the plurality of primers with respect to the reaction plate.

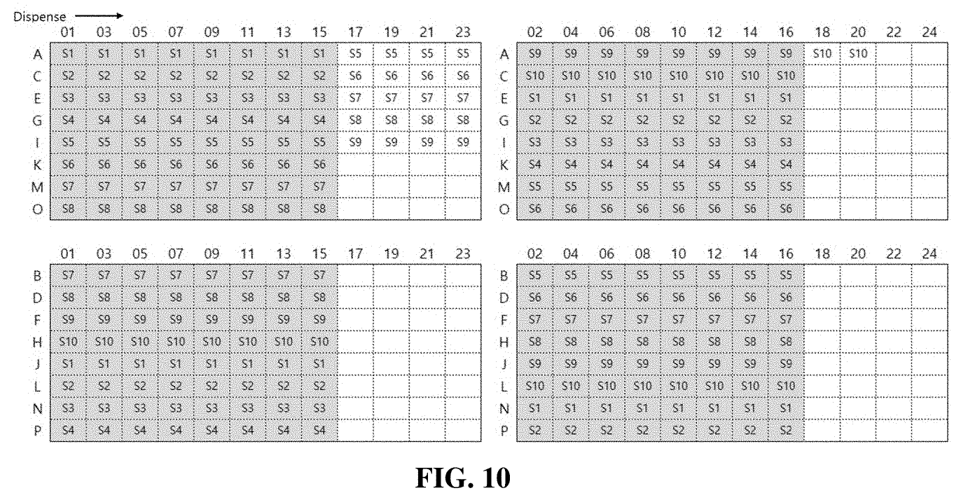

In an embodiment, arrangement of a plurality of samples with respect to a reaction plate may be performed corresponding to arrangement of primers. For example, once arrangement based on simultaneous dispensing counts and single dispensing counts is complete with respect to a primer group, the arrangement part 343 may be implemented such that the arrangement with respect to the samples is performed corresponding to the arrangement of the primers on the reaction plate as shown in FIG. 10.

In the meantime, the result of the arrangement of the samples and primers with respect to the reaction plate may be displayed to a user by being output to a user via a display 320 or by being transmitted to a user's terminal connected by wire or wireless communication.

In S460, the arrangement part 343 may arrange primers on a mixing plate based on the grouping result in S430. Here, the mixing plate is for mixing various reagents (for example, master mix, etc.) before dispensing the primers on the reaction plate and may include 96 wells (8 rows×12 columns), identical to the sample plate and the primer plate.

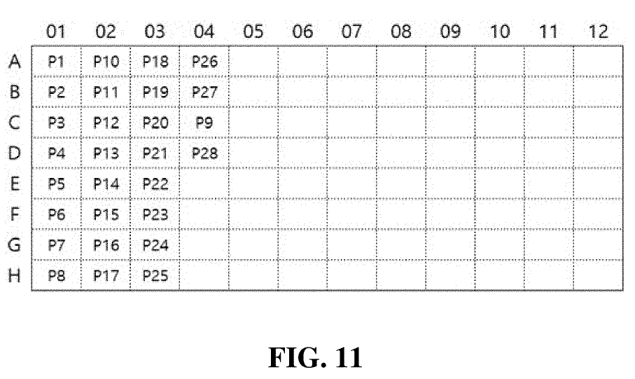

Here, the arrangement of the primers on the mixing plate may be identical to the arrangement of the primers on the virtual plate used for grouping in S430, as shown in FIG. 11, or may be in a different shape where only the disclosure location is horizontally moved.

In S470, the mapping part 344 may mutually map the arrangement of the samples between the sample plate and the reaction plate; and the arrangement of the primers among the primer plate, the mixing plate, and the reaction plate. Such mapping may be performed by mutually connecting the coordinates of the wells where the identical samples and primers are arranged in each plate.

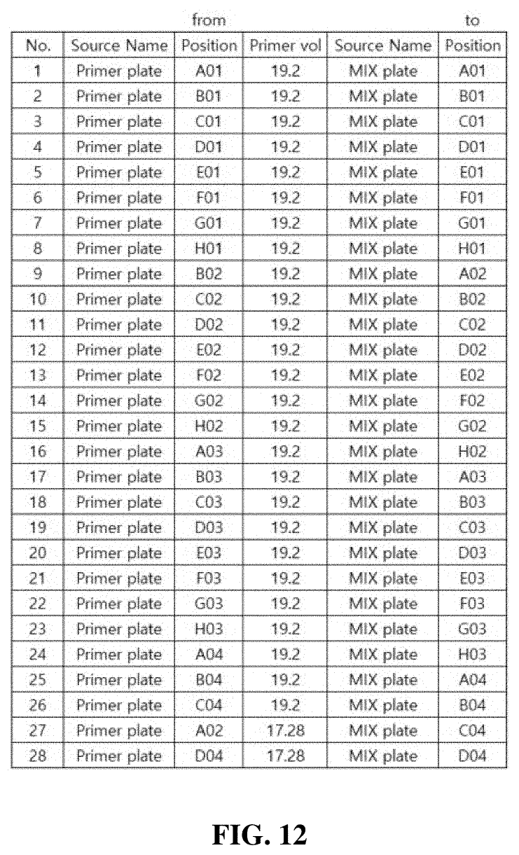

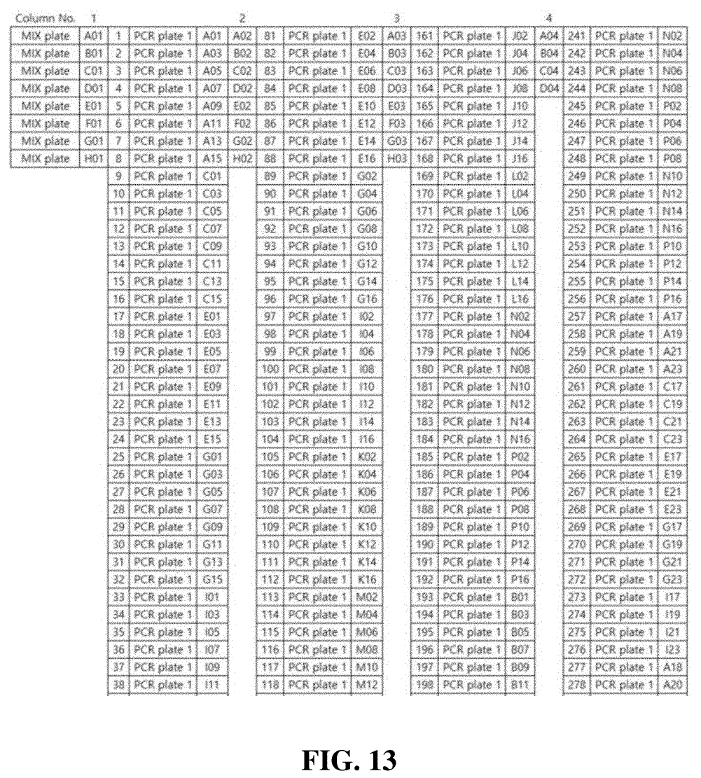

For example, the arrangement of the primers on the primer plate, the reaction plate and the mixing plate as shown in FIGS. 2B, 9 and 11, may be mutually mapped as shown in FIGS. 12 and 13. Here, FIG. 12 shows the mapping result between the primer plate and the mixing plate, and FIG. 13 shows the mapping result between the mixing plate and the reaction plate.

In an embodiment, once S470 is complete, an operation method 400 may further include storing at least one of the simultaneous dispensing counts and single dispensing counts calculated with respect to the sample and/or primer group and arrangement of the samples and primers on the reaction plate and the final mapping result in the storage part 330 upon the grouping result corresponding to the analysis information. The stored mapping result may be provided or transmitted to the user.

In an embodiment, the operation method 400 may further include calculating at least one of anticipated dispensing time, the number of replacement of dispensing nozzles, and moving distance of the dispensing nozzle when multi analysis is performed based on the mapping result. Such information may be stored in the storage part 330 along with the mapping result, etc.

In an embodiment, the operation method 400 may further include receiving user's edit request and, based thereon, editing the grouping of the samples or primers, the arrangement on the reaction plate and/or the arrangement on the mixing plate. As such, a user may edit the grouping of the samples or primers or the arrangement of the samples and the primers reflecting his or her preferences, actual experiment conditions, etc.

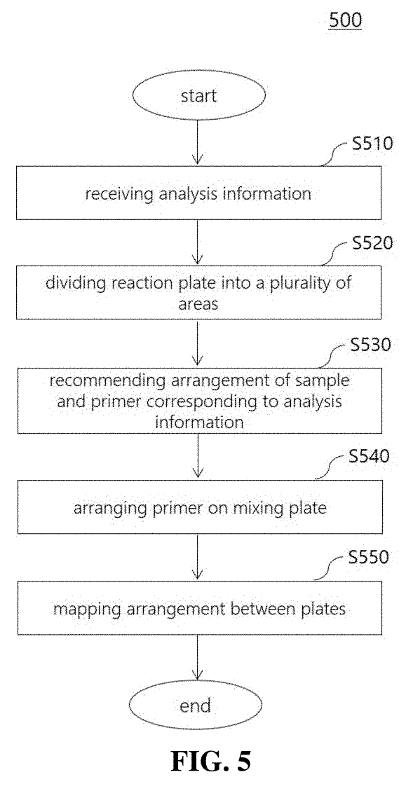

FIG. 5 shows an operation method of a dispenser mapping apparatus in an embodiment.

Referring to FIG. 5, a dispenser mapping apparatus 300 may auto recommend at least one arrangement of sample and primer. Here, S510, S520, S540 and S550 in FIG. 5 are identical to S410, S420, S460 and S470 of the operation method 400 in FIG. 4. Thus, what is repetitive will be omitted, and accordingly FIG. 4 may be referred to for further details.

In S530, a recommendation part 345 may recommend arrangement of sample and primer on a reaction plate suitable for analysis information in response to the analysis information it receives. That is, when the analysis information is received from the user, the recommendation part 345 may search the arrangement of the sample and the primer suitable for analysis information by searching the storage part 330 and provide it to the user.

In an embodiment, S530 may be performed based on a predetermined standard that includes at least one of dispensing time, the number of replacement of the dispensing nozzles and moving distance of dispensing nozzles. For example, a user may select at least one of dispensing time, the number of replacement of the dispensing nozzles and moving distance of dispensing nozzles as a recommendation standard, and the recommendation part 345 may detect one or more arrangements corresponding to sample input by a user and detection gene or primer of each sample, and recommend to the user the arrangement suitable for the standard selected by the user from the detected arrangements.

Meanwhile, the various embodiments described herein may be implemented by hardware, middleware, micro code, software and/or the combination thereof, for example, one or more application specific integrated circuits (ASICs), digital signal processors (DSP's), digital signal processing devices (DSPD's), programmable logic devices (PLD's), field programmable gate arrays (FPGA's), processors, controllers, microcontrollers, microprocessors, and other electronic units designed to perform functions disclosed herein and a combination thereof.

Also, for example, various embodiments may be recorded or encoded in computer readable medium that includes commands. The commands recorded or encoded in computer readable medium may cause programmable processor or other processor to, for example, perform methods when commands are executed. Computer readable medium may include computer storage medium. The storage medium may be an arbitrary usable medium that may be accessed by a computer. For example, such computer-readable medium ma include RAM, ROM, EEPROM, CD-ROM or other optical disc storage medium, magnetic disk storage medium or other magnetic storage device or other medium that can be used to store any desired program codes into commands or data structures which can be accessed by computer.

Such hardware, software, firmware, etc., may be implemented within a same device or separate devices to support various operations and functions described herein. Additionally, any element, unit, module, component, etc. that is described as a “. . . part” may be implemented together or individually as interoperable logic devices. Description of different features regarding modules, units, etc. are intended to emphasize different functional embodiments, and it does not necessarily mean that they need to be implemented by individual hardware or software components. Rather, functions related to one or more modules or units may be performed by individual hardware or software components or integrated within common or individual hardware or software components.

Although operations are depicted in drawings to have certain sequences, but it should not be understood to mean that such operations need to be performed in certain sequence or sequentially in order to achieve results. Also, it should not be understood to mean that all of the operations need to be performed in order to achieve results. In an arbitrary environment, multi-tasking and serial processing may be advantageous. Moreover, in the above-described embodiments, distinctions made with respect to various elements should not be understood to require such distinctions in all embodiments. It must be understood that the aforementioned elements can be generally integrated as single software products or packaged as multiple software products.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A method for operating a dispenser mapping apparatus for multi-analysis, the method comprising:
receiving analysis information, the analysis information including information relating to at least one of a plurality of samples, a detection gene for each of the plurality of samples, and one of a plurality of primers corresponding to the detection gene which are subject to analysis;
dividing a reaction plate into a plurality of areas; and
sequentially arranging the plurality of samples and the plurality of primers on the plurality of areas of the reaction plate.

2. The method of claim 1, wherein the step of the dividing into the plurality of areas is performed based on at least one of a maximum possible simultaneous dispensing count, an interval between dispensing nozzles, and an interval between wells of the reaction plate.

3. The method of claim 2, wherein at least one of rows and columns of each of the plurality of areas divided on the reaction plate is the same with the maximum possible simultaneous dispensing count.

4. The method of claim 1, further comprising:
grouping at least one of the plurality of samples and the plurality of primers into at least one group based on the analysis information,
wherein the step of the arranging of the plurality of samples and the plurality of primers is performed based on a result of the step of the grouping, and
wherein a number of maximum samples and the plurality of primers belonging to each of the at least one group is the same with a number of a maximum possible simultaneous dispensing count.

5. The method of claim 4, wherein the analysis information further includes information relating to an arrangement of the plurality of primers on a primer plate,
wherein the step of the grouping groups the plurality of primers into the at least one group by sequentially giving a priority to an arrangement sequence of the plurality of primers on the primer plate and a dispensing count of each of the plurality of primers.

6. The method of claim 4, further comprising:
calculating a simultaneous dispensing count with respect to at least one of the plurality of samples and the plurality of primers belonging to each of the at least one group,
wherein the step of the arranging of the plurality of samples and the plurality of primers is performed by sequentially arranging at least one of the plurality of samples and the plurality of primers belonging to each of the at least one group onto the plurality of areas of the reaction plate corresponding to the simultaneous dispensing count.

7. The method of claim 6,
wherein the step of the arranging the plurality of samples and the plurality of primers arranges a sample and a primer belonging to a group with a highest number of the simultaneous dispensing count with a priority.

8. The method of claim 4, further comprising:
calculating a simultaneous dispensing count and a single dispensing count with respect to at least one of the plurality of samples and the plurality of primers belonging to each of the at least one group,
wherein the step of the arranging the plurality of samples and the plurality of primers comprises:
sequentially arranging at least one of the plurality of samples and the plurality of primers belonging to each of the at least one group onto the plurality of areas of the reaction plate corresponding to the simultaneous dispensing count; and
when an arrangement according to the simultaneous dispensing count is completed, sequentially arranging at least one of the plurality of samples and the plurality of primers belonging to each of the at least one group onto remaining areas of the reaction plate based on the single dispensing count.

9. The method of claim 4, wherein the analysis information further includes information relating to an arrangement of the plurality of samples on a sample plate and an arrangement of the plurality of primers on a primer plate,
wherein the method further comprises:
arranging the plurality of primers on a mixing plate based on the result of the step of the grouping; and
mutually mapping:
a sample arrangement between the sample plate and the reaction plate, and
a primer arrangement among the primer plate, the mixing plate, and the reaction plate.

10. The method of claim 9, further comprising:
when the mapping is completed, storing at least one of the result of the step of the grouping, the simultaneous dispensing count, the single dispensing count, the plurality of samples with respect to the reaction plate, the arrangement of the plurality of primers, and the mapping result into a storage part corresponding to the analysis information.

11. The method of claim 1, further comprising recommending the plurality of samples suitable for the analysis information and the arrangement of the plurality of primers on the reaction plate according to a predetermined standard in response to receipt of the analysis information,
wherein the predetermined standard includes at least one of dispensing time, a number of replacement of dispensing nozzles, and moving distance of the dispensing nozzles.

12. A non-transitory computer readable recording medium recording a program to perform the method of claim 1.

13. A dispenser mapping apparatus for multi analysis comprising:
an input part receiving analysis information, the analysis information including information relating to at least one of a plurality of samples, a detection gene for each of the plurality of samples, and one of a plurality of primers corresponding to the detection gene which are subject to analysis; and
an arrangement part dividing a reaction plate into a plurality of areas and sequentially arranging the plurality of samples and the plurality of primers on the plurality of areas of the reaction plate.

14. The apparatus of claim 13, wherein the arrangement part divides the reaction plate into the plurality of areas based on at least one of a maximum possible simultaneous dispensing count, an interval between dispensing nozzles and an interval between wells of the reaction plate.

15. The apparatus of claim 14, wherein at least one of rows and columns of each of the plurality of areas of the reaction plate is the same with the maximum possible simultaneous dispensing count.

16. The apparatus of claim 13, further comprising a cluster part grouping at least one of the plurality of samples and the plurality of primers into at least one group based on the analysis information,
wherein the arrangement part performs an arrangement of the plurality of samples and the plurality of primers based on a result of the step of the grouping, and
wherein a number of maximum samples and the plurality of primers belonging to each of the at least one group is the same with a number of a maximum possible simultaneous dispensing count.

17. The apparatus of claim 16, wherein the analysis information further includes information relating to an arrangement of the plurality of primers on a primer plate,
wherein the cluster part groups the plurality of primers into the at least one group by sequentially giving a priority to an arrangement sequence of the plurality of primers on the primer plate and a dispensing count of each of the plurality of primers.

18. The apparatus of claim 16, further comprising a calculation part calculating a simultaneous dispensing count with respect to at least one of the plurality of samples and the plurality of primers belonging to each of the at least one group,
wherein the arrangement part sequentially arranges at least one of the plurality of samples and the plurality of primers belonging to each of the at least one group onto the plurality of areas of the reaction plate corresponding to the simultaneous dispensing count.

19. The apparatus of claim 18, wherein the arrangement part arranges a sample and a primer belonging to a group with a highest number of the simultaneous dispensing count with a priority.

20. The apparatus of claim 16, further comprising a calculation part calculating a simultaneous dispensing count and a single dispensing count with respect to at least one of the plurality of samples and the plurality of primers belonging to each of the at least one group,
wherein the arrangement part sequentially arranges at least one of the plurality of samples and the plurality of primers belonging to each of the at least one group onto the plurality of areas of the reaction plate corresponding to the simultaneous dispensing count, and, when an arrangement according to the simultaneous dispensing count is completed, sequentially arranges at least one of the plurality of samples and the plurality of primers belonging to each of the at least one group onto remaining areas of the reaction plate based on the single dispensing count.

21. The apparatus of claim 16,
wherein the analysis information further includes information relating to an arrangement of the plurality of samples on a sample plate and an arrangement of the plurality of primers on a primer plate,
wherein the apparatus further comprises a mapping part arranging the plurality of primers on a mixing plate based on the result of the step of the grouping and mutually mapping: a sample arrangement between the sample plate and the reaction plate; and a primer arrangement among the primer plate, the mixing plate, and the reaction plate.

22. The apparatus of claim 21, further comprising a storage part storing, when the mapping is completed, at least one of the grouping result, the simultaneous dispensing count, the single dispensing count, the plurality of samples with respect to the reaction plate, the arrangement of the plurality of primers, and the mapping result corresponding to the analysis information.

23. The apparatus of claim 13, further comprising a recommendation part recommending the plurality of samples suitable for the analysis information and the arrangement of the plurality of primers on the reaction plate according to a predetermined standard in response to receipt of the analysis information,
wherein the predetermined standard includes at least one of dispensing time, a number of replacement of dispensing nozzles, and moving distance of the dispensing nozzles.
